(19) United States
(12) Patent Application Publication
(10) Pub. No.: US 2021/0398653 A1
(43) Pub. Date: Dec. 23, 2021

(54) KEY IMAGE UPDATING MULTIPLE STACKS

(71) Applicant: FOVIA, INC., Palo Alto, CA (US)

(72) Inventors: Kevin KREEGER, Palo Alto, CA (US); David WILKINS, Allison Park, PA (US)

(73) Assignee: FOVIA, INC., Palo Alto, CA (US)

(21) Appl. No.: 17/349,652

(22) Filed: Jun. 16, 2021

Related U.S. Application Data
(60) Provisional application No. 63/040,402, filed on Jun. 17, 2020.

(51) Int. Cl. G16H 30/20; G16H 30/40; G16H 50/20; G06F 16/54

(52) U.S. Cl. CPC G16H 30/20; G06F 16/54; G16H 50/20; G16H 30/40

(57) ABSTRACT

A process for key image updating multiple image stacks includes obtaining a first key image list comprising at least one first key image corresponding to a first stack of medical images and obtaining a second key image list comprising at least one second key image corresponding to a second stack of medical images. The first key images are then mapped to the corresponding second key images such that in response to selection of one of the first key images of the first stack the process can map and display the selection of the first key image and the corresponding second key image of the second stack of medical images. In some examples, more than two stacks (e.g., 3 or more) of medical images can be mapped or synchronized in this fashion.

### CROSS-REFERENCE TO RELATED APPLICATIONS

This application claims the benefit of U.S. Provisional Patent Application No. 63/040,402, entitled, “KEY IMAGE UPDATING MULTIPLE STACKS,” filed Jun. 17, 2020, the content of which is hereby incorporated by reference in its entirety for all purposes.

### FIELD

This relates generally to medical images, and in particular to key image updating with respect to multiple stacks of medical images.

### SUMMARY

According to one embodiment, two (or more) key image lists are created, each having its own key images for one stack. The key image lists are then synchronized or mapped to allow a user to quickly navigate between the key images of each stack.

In one example, a method for key image updating multiple image stacks includes obtaining a first key image list comprising at least one first key image corresponding to a first stack of medical images and obtaining a second key image list comprising at least one second key image corresponding to a second stack of medical images. The first key images are then mapped to the corresponding second key images such that in response to selection of one of the first key images of the first stack the process can map and display the selection of the first key image and the corresponding second key image of the second stack of medical images. In some examples, more than two stacks (e.g., 3 or more) of medical images can be mapped or synchronized in this fashion.

The first key image list and the second key image list can be separately stored lists or merged into a common, single list of key images. The first key image list and the second key image list include images of a stack of medical images having findings of identified areas of interest. For example, the images may be processed by a computer aided detection algorithm, artificial intelligence algorithm, machine learning algorithm, or the like, for identifying areas of interest in medical images.

In other embodiments, computer readable storage medium and systems for carrying out described processes are provided.

### BRIEF DESCRIPTION OF THE DRAWINGS

FIG. 1 illustrates various embodiments for key image updating multiple stacks of medical images.

FIG. 2 illustrates an exemplary process for mapping key images of multiple stacks of medical images.

FIG. 3 illustrates an exemplary system 100 for visualization of medical images.

### DETAILED DESCRIPTION

There are many types of AI algorithms to assist radiologists in interpreting medical imaging studies. There are algorithms to assist in actual reading of the scanned images, there are algorithms to automatically find prior imaging studies of the patient, there are algorithms to make predictions based on other patient information than just the images, there are algorithms that help scheduling in the scanner rooms, there are algorithms that assist in deciding what scans should be done and many more. This disclosure is related to efficiently assisting the radiologist in reading the medical images.

The AI algorithms used to help detect or interpret disease and can be further subdivided into several groups. There are algorithms that classify disease, there are algorithms that measure structures in the images, there are algorithms that segment structures in the images, and many more.

This disclosure concerns algorithms that detect or classify disease or areas of interest in the images. Furthermore, this disclosure will address algorithms commonly knows a CAD (Computer Aided Detection) where the algorithm highlights multiple suspicious areas of abnormalities in the images.

In a radiology setting, it is advantageous to provide a mechanism that allows the user to more efficiently navigate the abnormalities, or findings, in the stack of images and quickly advanced through these findings to accept, reject, or modify each of these findings. Depending on the type of medical imaging modality, the multiple findings may not be visible all at once. The physician must scroll up and down through the image stack (set of images) searching for the findings. It should be noted that a given study may have one or more stacks of images, where each stack may or may not have been processed by an AI algorithm.

There currently exists a standard practice where certain images that are important or flagged are marked as a key image. In some cases there may be multiple key images in each stack of images, and the user can quickly navigate between the key images (to navigate to the next finding). However, a limitation of key images in conventional systems is that it only points to one single image or a single stack of images, so when navigating to the next finding, only one stack will be updated.

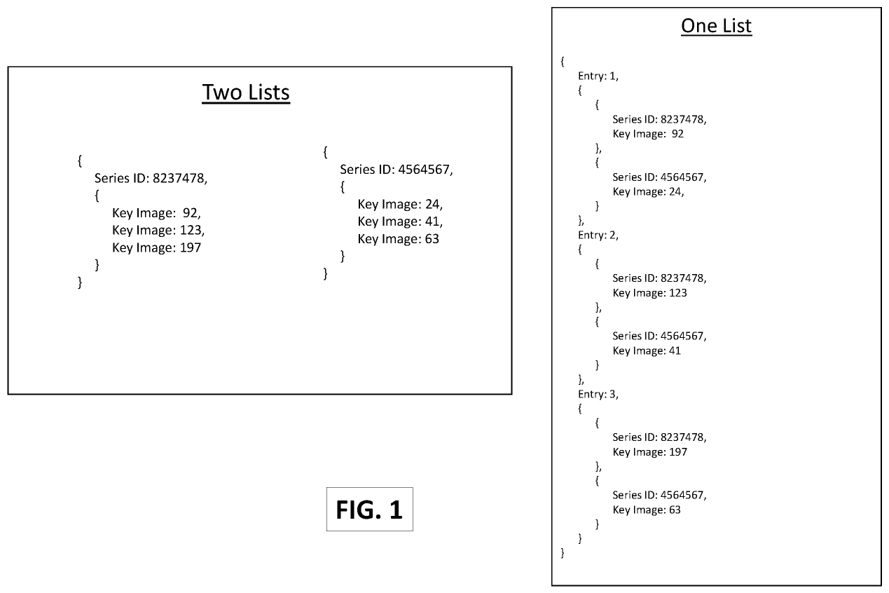

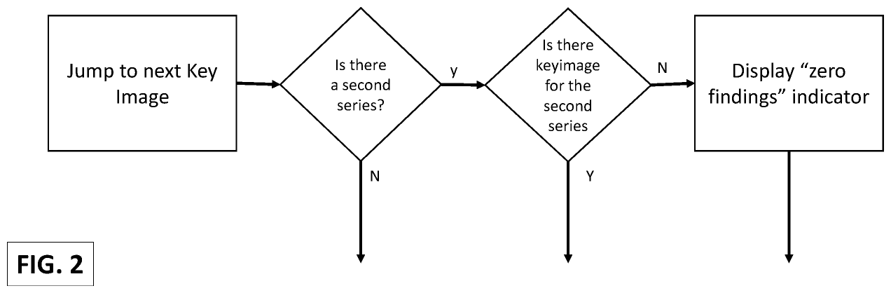

With reference to FIGS. 1 and 2, one aspect of the invention establishes a relationship between two or more stack of images, such that when the user navigates to the key image in a first stack, the other stack(s) automatically advance to the same corresponding key image (or at least provide an indication of the next key image in the other stack(s)) or provides an indication that there is no corresponding key image. Sometimes there is a secondary stack of images which signify something else about the case, and specifically about the key image in the other stack of images. Sometimes it contains extra information about the abnormality associated with the key image, such as measurement information, prior study information, etc.. It is therefore advantageous to be able to navigate multiple stacks to the appropriate image in their corresponding stack of image when jumping to the next key image. Sometimes the image will be the same index in both stacks, sometimes it will not.

In one example, key images from multiple stacks can be associated or mapped by creating two (or more) key image lists, each having its own key images for one stack. In this example, the key image lists are synchronized, and can utilize the constructs in the current standardized objects (such as DICOM Key Image Objects). An example of two key image list are illustrated on the left side of FIG. 1.

In another example, key images from multiple stacks can be added to one key image list referring two (or more) image indexes for each entry, one for each stack of images. This will differ from current standardized objects for storing a key image list, e.g., some modification thereto or through the use of private tags to establish this relationship. An example of a single key image list is illustrated on the right side of FIG. 1.

FIG. 2 illustrates an exemplary process for displaying and navigating key images that have been mapped as described herein. The exemplary process includes a request to move to the next key images, e.g., from a user input indicating a request to jump to the next key image to view a finding. The system may then obtain the mapping of key images between multiple lists corresponding to each stack of images or a merged, common list as described, in order to display or navigate the user to the next key image in 2 or more stack of images. Accordingly, in response to a user navigation to a key image, the system determines if there is a second (or third) stack of images. If so, the process further determines if there is a corresponding key image in the stack to display. If there is a corresponding key image they process can display the corresponding key image, and if there is not, the system can display an indication of no findings in the second (or third) stack of images.

Various embodiments described herein may be carried out by computer devices, medical imaging systems, and computer-readable medium comprising instructions for carrying out the described methods.

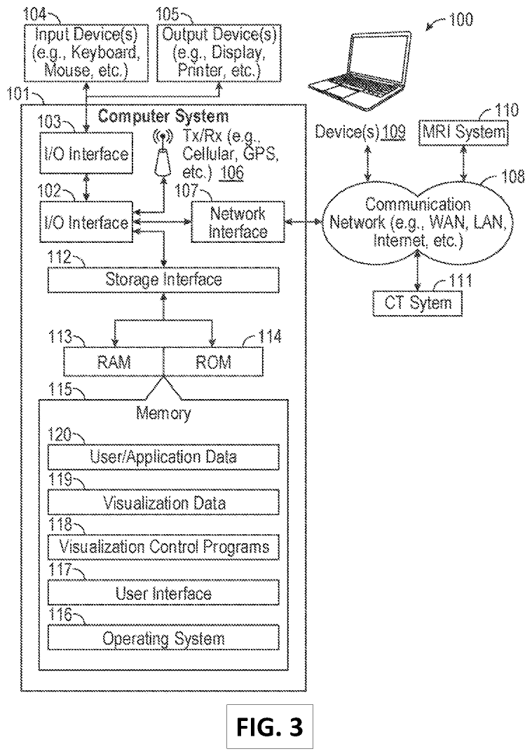

FIG. 3 illustrates an exemplary system 100 for visualization of medical images, consistent with some embodiments of the present disclosure. System 100 may include a computer system 101, input devices 104, output devices 105, devices 109, Magnet Resonance Imaging (MRI) system 110, and Computer Tomography (CT) system 111. It is appreciated that one or more components of system 100 can be separate systems or can be integrated systems. In some embodiments, computer system 101 may comprise one or more central processing units (“CPU” or “processor(s)”) 102. Processor(s) 102 may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 102 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) 102 may be disposed in communication with one or more input/output (I/O) devices via I/O interface 203. I/O interface 103 may employ communication protocols/methods such as, without limitation, audio, analog, digital, monaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 103, computer system 101 may communicate with one or more I/O devices. For example, input device 104 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. Output device 105 may be a printer, fax machine, video display (e.g., cathode ray tube (CRT), liquid crystal display (LCD), light-emitting diode (LED), plasma, or the like), audio speaker, etc. In some embodiments, a transceiver 106 may be disposed in connection with the processor(s) 102. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, processor(s) 102 may be disposed in communication with a communication network 108 via a network interface 107. Network interface 107 may communicate with communication network 108. Network interface 107 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication network 108 may include, without limitation, a direct interconnection, local area network (LAN), wide area network (WAN), wireless network (e.g., using Wireless Application Protocol), the Internet, etc. Using network interface 107 and communication network 108, computer system 101 may communicate with devices 109. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, computer system 101 may itself embody one or more of these devices.

In some embodiments, using network interface 107 and communication network 108, computer system 101 may communicate with MRI system 110, CT system 111, or any other medical imaging systems. Computer system 101 may communicate with these imaging systems to obtain images for display. Computer system 101 may also be integrated with these imaging systems.

In some embodiments, processor 102 may be disposed in communication with one or more memory devices (e.g., RAM 213, ROM 214, etc.) via a storage interface 112. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system 116, user interface 117, medical visualization program 118, visualization data 119 (e.g., tie data, registration data, colorization, etc.), user/application data 120 (e.g., any data variables or data records discussed in this disclosure), etc. Operating system 116 may facilitate resource management and operation of computer system 101. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 117 may facilitate display, execution, interaction, manipulation, or operation of program components through textual or graphical facilities. For example, user interfaces may provide computer interaction interface elements on a display system operatively connected to computer system 101, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, computer system 101 may implement medical imaging visualization program 118 for controlling the manner of displaying medical scan images. In some embodiments, computer system 101 can implement medical visualization program 118 such that the plurality of images are displayed as described herein.

In some embodiments, computer system 101 may store user/application data 120, such as data, variables, and parameters (e.g., one or more parameters for controlling the displaying of images) as described herein. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination

It should be noted that, despite references to particular computing paradigms and software tools herein, the computer program instructions with which embodiments of the present subject matter may be implemented may correspond to any of a wide variety of programming languages, software tools and data formats, and be stored in any type of volatile or nonvolatile, non-transitory computer-readable storage medium or memory device, and may be executed according to a variety of computing models including, for example, a client/server model, a peer-to-peer model, on a stand-alone computing device, or according to a distributed computing model in which various of the functionalities may be effected or employed at different locations. In addition, references to particular algorithms herein are merely by way of examples. Suitable alternatives or those later developed known to those of skill in the art may be employed without departing from the scope of the subject matter in the present disclosure.

It will be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

What is claimed is:

1. A computer-implemented method for key image updating multiple image stacks, the method comprising:
obtaining a first key image list comprising at least one first key image corresponding to a first stack of medical images;
obtaining a second key image list comprising at least one second key image corresponding to a second stack of medical images, wherein the at least one first key image is mapped to the at least one second key image; and
in response to a selection of one of the at least one first key image of the first stack of medical images for display, displaying the selection of the one of the at least one first key image of the first stack of medical images and a corresponding one of the at least one second key image of the second stack of medical images based on the mapping.

2. The method of claim 1, further comprising:
obtaining a third key image list comprising at least one third key image corresponding to a third stack of medical images, wherein the at least one third key image is mapped to the at least one first key image and/or the at least one second key image; and
in response to a selection of one of the at least one first key image of the first stack of medical images for display, displaying the selection of the one of the at least one first key image of the first stack of medical images and a corresponding one of the at least one second key image of the second stack of medical images and/or one of the at least third key image of the third stack of medical images based on the mapping.

3. The method of claim 1, wherein the first key image list and the second key image list are separately stored lists.

4. The method of claim 1, wherein the first key image list and the second key image list are included in a common list of key images.

5. The method of claim 1, wherein the first key image list and the second key image list include images of a stack of medical images having findings of identified areas of interest.

6. The method of claim 5 , wherein the stack of images was analyzed with a computer aided detection algorithm for identifying areas of interest in medical images.

7. The method of claim 5, wherein the stack of images was analyzed with an artificial intelligence algorithm for identifying areas of interest in medical images.

8. The method of claim 5, wherein the stack of images was analyzed with a machine learning algorithm for identifying areas of interest in medical images.

9. A computer readable storage medium, comprising instructions for:
obtaining a first key image list comprising at least one first key image corresponding to a first stack of medical images;
obtaining a second key image list comprising at least one second key image corresponding to a second stack of medical images, wherein the at least one first key image is mapped to the at least one second key image; and
in response to a selection of one of the at least one first key image of the first stack of medical images for display, displaying the selection of the one of the at least one first key image of the first stack of medical images and a corresponding one of the at least one second key image of the second stack of medical images based on the mapping.

10. The computer readable storage medium of claim 9, wherein the first key image list and the second key image list are separately stored lists.

11. The computer readable storage medium of claim 9, wherein the first key image list and the second key image list are included in a common list of key images.

12. The computer readable storage medium of claim 9, wherein the first key image list and the second key image list include images of a stack of medical images having findings of identified areas of interest.

13. The computer readable storage medium of claim 12, wherein the stack of images was analyzed with a computer aided detection algorithm for identifying areas of interest in medical images.

14. The computer readable storage medium of claim 12, wherein the stack of images was analyzed with an artificial intelligence algorithm for identifying areas of interest in medical images.

15. The computer readable storage medium of claim 12, wherein the stack of images was analyzed with a machine learning algorithm for identifying areas of interest in medical images.

16. A system comprising a processor and memory, the memory storing instructions for
obtaining a first key image list comprising at least one first key image corresponding to a first stack of medical images;
obtaining a second key image list comprising at least one second key image corresponding to a second stack of medical images, wherein the at least one first key image is mapped to the at least one second key image; and
in response to a selection of one of the at least one first key image of the first stack of medical images for display, displaying the selection of the one of the at least one first key image of the first stack of medical images and a corresponding one of the at least one second key image of the second stack of medical images based on the mapping.

17. The system of claim 16, wherein the first key image list and the second key image list are separately stored lists.

18. The system of claim 16, wherein the first key image list and the second key image list are included in a common list of key images.

19. The system of claim 16, wherein the first key image list and the second key image list include images of a stack of medical images having findings of identified areas of interest.

20. The system of claim 19, wherein the stack of images was analyzed with a computer aided detection algorithm for identifying areas of interest in medical images.

21. The system of claim 19, wherein the stack of images was analyzed with an artificial intelligence algorithm for identifying areas of interest in medical images.

22. The system of claim 19, wherein the stack of images was analyzed with a machine learning algorithm for identifying areas of interest in medical images.
